(19) United States
(12) Patent Application Publication
(10) Pub. No.: US 2026/0047799 A1
(43) Pub. Date: Feb. 19, 2026

(54) Body-Worn Physiological Monitoring Device and System for Genital Region Applications

(71) Applicant: Dajuan Williams, Detroit, MI (US)

(72) Inventor: Dajuan Williams, Detroit, MI (US)

(21) Appl. No.: 19/370,688

(22) Filed: Oct. 27, 2025

Related U.S. Application Data
(63) Continuation-in-part of application No. 19/238,431, filed on Jun. 14, 2025.
(63) Continuation-in-part of application No. 19/260,509, filed on Jul. 6, 2025.
(60) Provisional application No. 63/732,328, filed on Aug. 5, 2024.
(60) Provisional application No. 63/778,945, filed on Mar. 27, 2025.

(51) Int. Cl. A61B 5/00

(52) U.S. Cl. CPC A61B 5/4393; A61B 5/0022; A61B 5/4368; A61B 5/4824; A61B 5/7282; A61B 2560/0242; A61B 2562/0219; A61B 2562/0271

(57) ABSTRACT

A body-worn electronic device is disclosed for monitoring physiological and environmental parameters in a genital region of a user. The device includes a housing configured for internal or external placement and incorporating one or more touch, temperature, and ambient-light sensors coupled to a microcontroller. The microcontroller establishes baseline values following a stabilization period, detects concurrent threshold deviations to classify confirmed events, and initiates data recording. A communication module having a wireless transceiver transmits event data to a paired mobile device or stores data locally when offline. The housing is formed of a flexible, biocompatible material that conforms to anatomical surfaces, improving sensor accuracy and comfort. The system enables continuous or periodic monitoring while preserving privacy through encrypted, consent-based operation

### BACKGROUND

The challenge of establishing behavioral transparency in monogamous relationships has persisted across cultures and generations. Traditional methods of confirming physical fidelity, such as direct confrontation, surveillance, or the engagement of third-party investigators, are often invasive, unreliable, and emotionally destabilizing. These approaches frequently produce incomplete or inaccurate information and can themselves contribute to breakdowns in trust and communication.

In the United States, suspicion of infidelity is a major driver of conflict within personal relationships and legal disputes, giving rise to significant expenditures on private investigators, litigation, and associated services. Estimates suggest that such expenditures amount to hundreds of millions of dollars annually. Despite the prevalence of these costs, existing solutions do not adequately address the need for discreet, objective, and verifiable methods of assessing physical fidelity.

Accordingly, there remains a need for improved systems that can provide reliable, technology-based data to support transparency in intimate relationships without reliance on invasive or adversarial practices.

### SUMMARY

The present disclosure relates to a body-worn electronic device configured to monitor physiological and environmental parameters when mounted on, around, or within a genital region of a user. The device comprises a housing incorporating multiple sensing components, including at least one touch sensor (such as a capacitive, resistive, or piezoelectric sensor) operable to detect physical contact; a temperature sensor; and an ambient light sensor configured to measure surrounding illumination levels. A microcontroller having a processor, memory, and input/output circuitry is programmed to receive and process data from the sensors, compare the data to one or more predefined thresholds stored in the memory, and initiate an event cycle in response to determining that data from at least two anomalies of the sensors exceed respective thresholds. During the event cycle, the microcontroller executes programmed instructions for data acquisition, applies timestamps, and generates an outbound data packet.

The device further includes a communication module comprising a wireless transceiver, memory, and controller configured to establish a short-range wireless link (e.g., Bluetooth Low Energy) with a paired mobile device. The communication module transmits event data generated by the microcontroller to the paired device, stores data locally when the connection is unavailable, and automatically transmits stored data upon reconnection. A power supply is integrated within the housing to energize the electronic components, enabling autonomous operation.

In certain embodiments, the device further includes an inertial measurement unit (IMU), a geolocation module for satellite-based position determination, and/or an onboard camera configured to capture still images or video sequences upon detection of a confirmed event. The housing may be adapted for vaginal insertion or mounting adjacent to the penis or scrotum, with the temperature sensor positioned to obtain accurate core body temperature measurements. The system can record physiological or behavioral data associated with sexual activity, fertility cycles, or urogenital health.

Associated methods of operation include initializing baseline sensor values, continuously sampling sensor signals, detecting and classifying anomalies, and activating the camera or data-logging functions when concurrent or temporally proximate anomalies are classified as confirmed events. The methods may further provide for secure data handling, including encryption, temporary storage, and cloud transmission through a paired smartphone. Additional embodiments permit user-initiated neutral logging for voluntary device removal, maintenance of chronological data integrity across offline periods, and operation only after mutual digital consent by two users through a system interface.

### BRIEF DESCRIPTION OF THE DRAWINGS

FIG. 1 is a peripheral view of a body-worn device configured for use in a female subject, illustrating representative placement of the housing within or adjacent to a vaginal canal.

FIG. 2 is a schematic view of a body-worn device configured for use in a female subject, illustrating representative placement of the housing within or adjacent to a vaginal canal and showing, in partial transparency, internal electronic components including a microcontroller, communication module, and power supply together with external temperature, touch, and ambient-light sensors.

FIG. 3 is a schematic view of a body-worn device configured for use in a male subject, illustrating representative placement of the housing on or around a penile or scrotal region and showing, in partial transparency, the arrangement of internal components and external sensor locations.

FIG. 4 is a functional block diagram of the electronic architecture of the body-worn device, showing operative connections among the touch, temperature, and ambient-light sensors, the microcontroller, the communication module, the power supply, and an external mobile or cloud device.

FIG. 5 is a flowchart illustrating an example firmware routine executed by the microcontroller, including initialization, stabilization delay, baseline averaging, event detection, data transmission, and hysteresis intervals.

### DETAILED DESCRIPTION OF THE INVENTION

The following detailed description illustrates embodiments of the present disclosure. These embodiments are described in sufficient detail to enable a person of ordinary skill in the art to practice these embodiments without undue experimentation. It should be understood, however, that the embodiments and examples described herein are given by way of illustration only, and not by way of limitation. Various substitutions, modifications, additions, and rearrangements may be made that remain potential applications of the disclosed techniques. Therefore, the description that follows is not to be taken as limiting on the scope of the appended claims. In particular, an element associated with a particular embodiment should not be limited to association with that particular embodiment but should be assumed to be capable of association with any embodiment discussed herein.

Unless otherwise defined, all technical and scientific terms used herein have the same meaning as commonly understood by one of ordinary skill in the art. In case of conflict, the present disclosure, including any definitions provided herein, shall control. The materials, methods, and examples disclosed herein are illustrative only and are not intended to be limiting. Methods and materials similar or equivalent to those described herein can be used in the practice or testing of the disclosed invention. All publications, patent applications, patents, and other references mentioned herein are incorporated by reference in their entirety, to the extent that they do not conflict with the disclosure provided in this document. As used in this application, the terms “comprise(s),” “include(s),” “having,” “has,” “can,” “contain(s),” and variants thereof are intended to be open-ended transitional phrases or terms that do not preclude the presence of additional elements or steps. The singular forms “a,” “an,” and “the” also include plural forms unless the context clearly dictates otherwise. The present disclosure also contemplates embodiments comprising, consisting of, or consisting essentially of the elements or features described herein, whether explicitly set forth or not.

### REFERENCE NUMERALS

Touch and/or pressure sensor—110
Ambient-light sensor—120
Temperature sensor—130
Onboard camera—140
Microcontroller—150
Communication module—160
Paired mobile device—200
Cloud server—210
Power supply—400
Female body-worn device embodiment—500
Male body-worn device embodiment—600

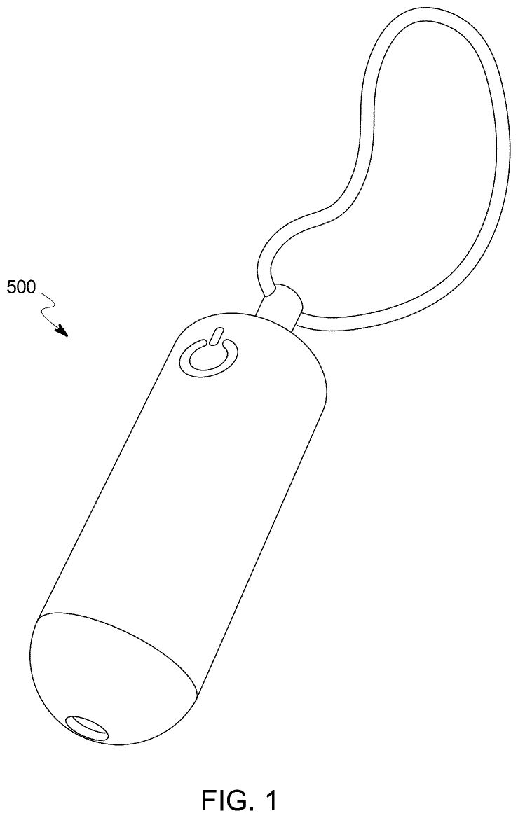

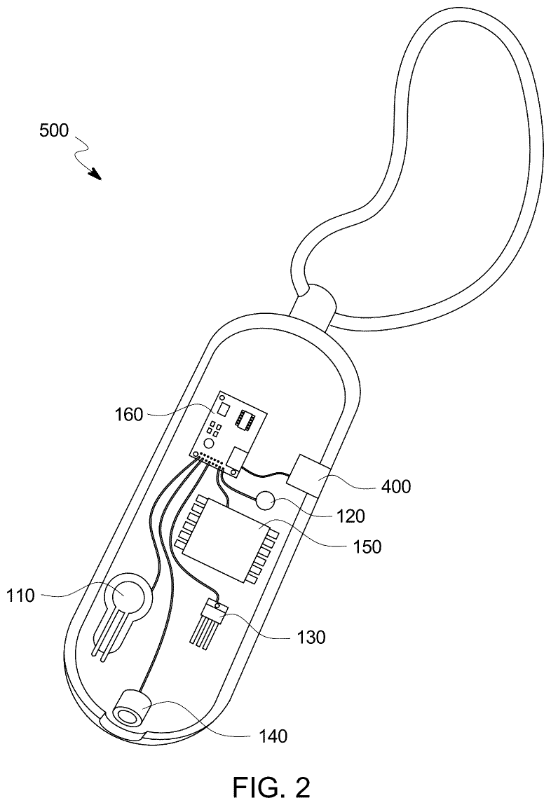

Referring now to FIG. 1, a female body-worn device (500) is shown configured for insertion within or adjacent to a vaginal canal of a user. The figure illustrates the overall external configuration of the housing, which is shaped and dimensioned for comfortable placement within the genital region. The housing is formed of a biocompatible, non-porous material such as medical-grade silicone or thermoplastic elastomer and may include surface features that promote stability and comfort during wear. Internal electronic components are contained within the housing and are described in greater detail with reference to FIG. 2.

Referring to FIG. 2, the internal architecture of the female body-worn device (500) is shown in greater detail. The touch/pressure sensor (110) detects mechanical contact with adjacent tissue; the ambient-light sensor (120) monitors illumination levels to detect insertion or removal; and the temperature sensor (130) monitors body temperature. The onboard camera (140), when present, is activated by the microcontroller (150) upon detection of a confirmed event. The communication module (160) provides a short-range wireless link, such as Bluetooth Low Energy (BLE), with a paired mobile device. The power supply (400) energizes the microcontroller and communication module, enabling autonomous operation.

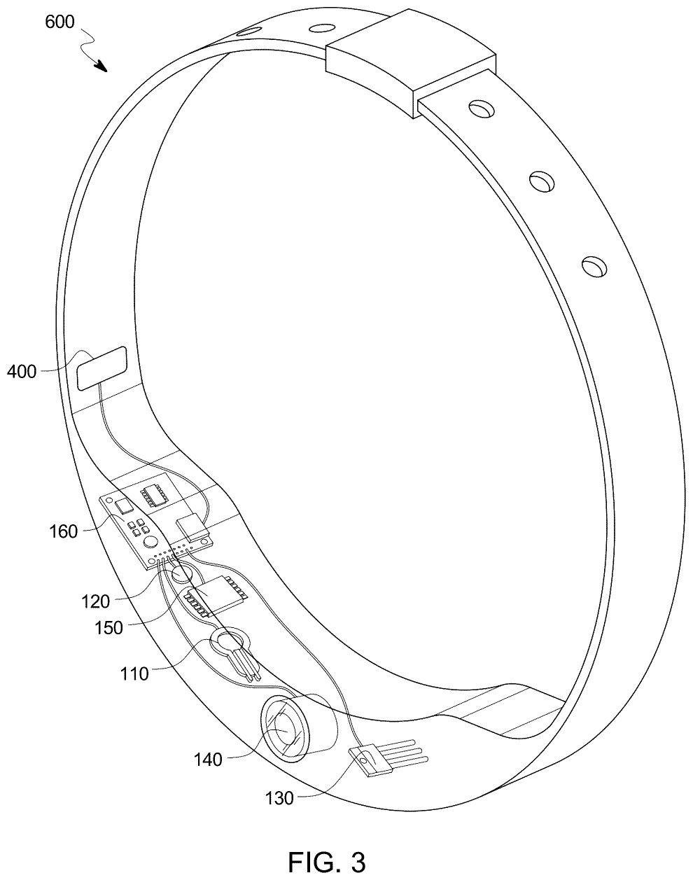

As shown in FIG. 3, a male body-worn device (600) includes corresponding components: the touch/pressure sensor (110), ambient-light sensor (120), temperature sensor (130), onboard camera (140), microcontroller (150), communication module (160), and power supply (400), all enclosed within a flexible, biocompatible housing. The housing may be configured as a sheath, ring, or wrap to be mounted on or around the penis or scrotum, ensuring stable sensor contact and accurate data acquisition.

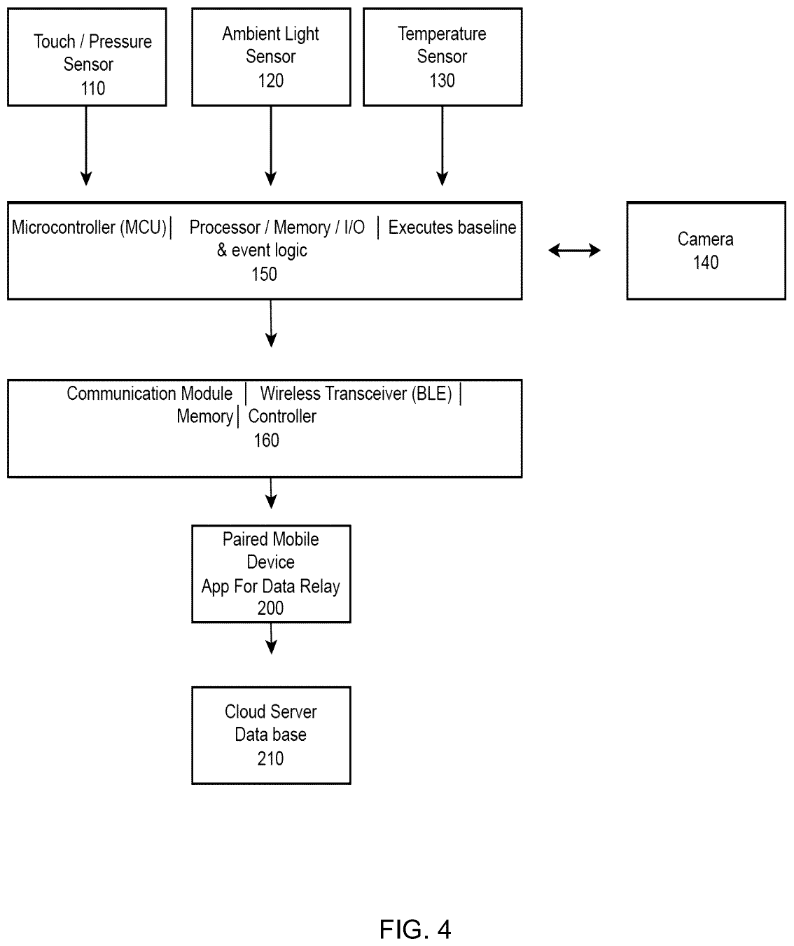

Referring to FIG. 4, the system architecture illustrates operative connections among the touch and/or pressure sensor (110), ambient-light sensor (120), temperature sensor (130), and, when present, the onboard camera (140). Each of these sensing elements provides respective analog or digital signals to the microcontroller (150). The microcontroller (150) processes the received data, classifies anomalies, and generates event packets transmitted to the communication module (160). The communication module (160) establishes a short-range wireless connection with a paired mobile device (200), which relays the encrypted data to a cloud server (210) for secure storage and analysis. The figure thereby depicts both internal signal flow between the sensing components and the microcontroller, and external data transmission through the communication and network chain.

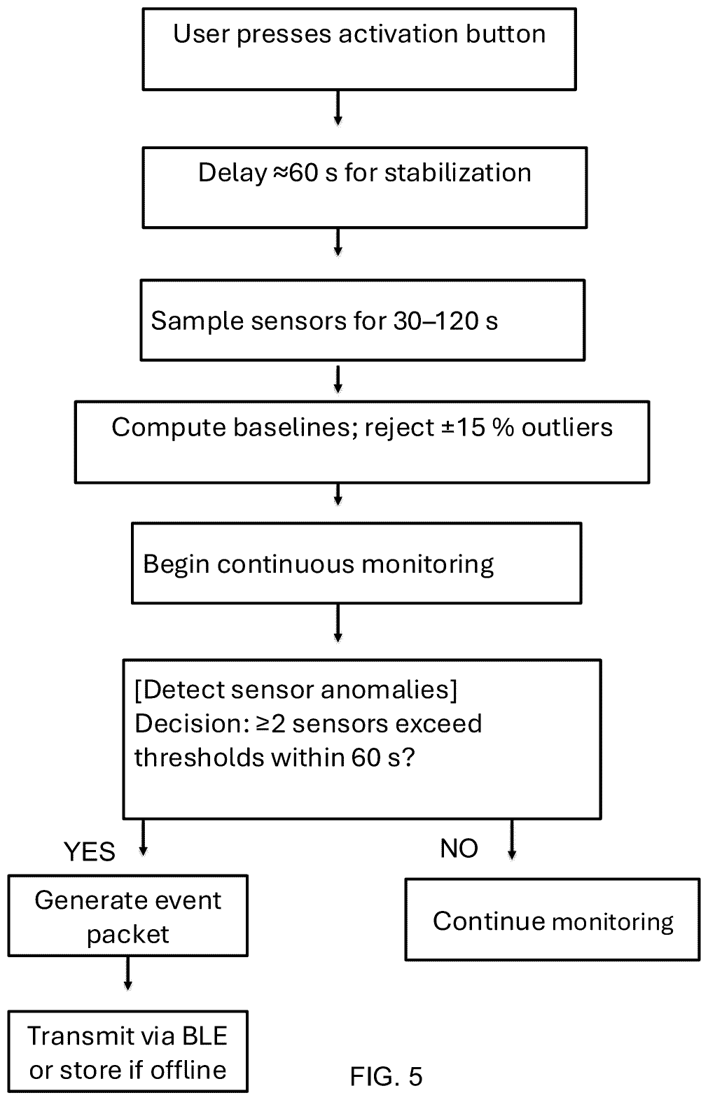

FIG. 5 depicts a flowchart illustrating an example firmware routine executed by the microcontroller, including initialization, stabilization delay, baseline averaging, event detection, data transmission, and hysteresis intervals. The microcontroller continuously monitors the outputs of the touch/pressure sensor, temperature sensor, and ambient-light sensor to classify anomalies and trigger event cycles as described herein.

In one embodiment, a body-worn device is provided for detecting and recording environmental and physiological changes when mounted within or adjacent to a genital region of a user. The device comprises a housing dimensioned and shaped for secure placement either internally or externally relative to the genital region. The housing may be formed from a biocompatible, non-porous material such as medical-grade silicone, polyether-ether-ketone (PEEK), or thermoplastic elastomer, and may include sealing features or coatings that render the internal electronics moisture-resistant.

The housing supports a plurality of sensors. At least one touch sensor is disposed on or near the exterior surface of the housing. The touch sensor may include any of a capacitive, resistive, or piezoelectric force-sensing element, each operable to detect contact between the housing and adjacent tissue or to sense variations in applied pressure. A capacitive touch sensor detects proximity or contact through changes in capacitance between conductive electrodes. A resistive touch sensor measures pressure through a change in resistance between conductive layers when compressed. A piezoelectric sensor produces a voltage proportional to applied mechanical stress and may be used to identify transient pressure changes or movement of the housing.

A temperature sensor is positioned within the housing in thermal communication with the body surface. The temperature sensor may be a thermistor, thermocouple, or solid-state temperature-sensing element. The microcontroller continuously samples the temperature and compares it with a baseline value stored in memory. When the temperature increases or decreases by 1.8° F. or more within ten seconds or less, the microcontroller classifies the change as a flagged temperature anomaly. In some embodiments a full event cycle is initiated only when at least one additional sensor channel exceeds its threshold within the confirmation window.

An ambient-light sensor is further disposed on the housing and oriented to detect illumination levels surrounding the device. The light sensor may include a photodiode or phototransistor sensitive to visible or near-infrared wavelengths. The microcontroller compares measured illumination to a baseline stored in memory. When illumination increases or decreases beyond a predefined threshold relative to the baseline, the change is interpreted as removal, insertion, or environmental interference, and the microcontroller flags a single-sensor anomaly. In some embodiments the microcontroller initiates an event cycle only upon classification of a confirmed event in which two or more anomalies or sensor channels exceed their respective thresholds within a defined temporal window.

A microcontroller contained within the housing includes a processor, non-volatile memory, volatile working memory, and input/output circuitry. The microcontroller executes instructions stored in memory to receive analog or digital signals from the touch, temperature, and ambient light sensors, to filter and digitize those signals, and to compare each processed value against a corresponding predefined threshold stored in memory. When the microcontroller detects that data from two or more sensors concurrently or sequentially exceed their respective thresholds, it classifies the condition as a confirmed event and initiates an event cycle. During the event cycle, the microcontroller collects synchronized data streams from the active sensors, applies internal timestamps to each data record, and assembles the resulting information into an outbound transmission packet for communication to the external device. The thresholds may represent absolute magnitudes, rates of change, or temporal deviations from baseline values, thereby allowing multi-sensor correlation to confirm genuine environmental or physiological events while minimizing false positives due to transient noise or minor fluctuations. The outbound transmission packet may include sensor identifiers, sequence numbers, and checksums to ensure chronological integrity and data authenticity.

A communication module comprising a wireless transceiver, controller, and local memory is electrically coupled to the microcontroller. The transceiver may employ Bluetooth Low Energy (BLE) or another short-range wireless protocol to establish a secure connection with a paired mobile device. The communication module transmits event data generated by the microcontroller to the paired device and temporarily stores the data locally when the wireless connection is unavailable. When connectivity is restored, the module automatically retrieves and forwards the stored data to the mobile device.

A power supply is integrated within the housing and provides electrical energy to the microcontroller and communication module. The power supply may include a rechargeable lithium-polymer battery or other miniature energy source and may optionally be recharged inductively through the housing wall.

The microcontroller and communication module cooperate to enable autonomous operation of the body-worn device. When the device is disconnected from the external mobile device, event data are stored in local memory, preserving chronological order and timestamps. Upon re-establishment of the wireless link, the stored data are transmitted automatically without user intervention. The described configuration thereby provides continuous monitoring of contact, temperature, and illumination conditions and ensures that event data are reliably captured, retained, and transmitted even in the absence of immediate network connectivity.

The requirement that data from two or more sensors exceed their respective thresholds before initiating an event cycle provides enhanced reliability and significantly reduces false-positive detections. Single-sensor anomalies may result from transient electrical noise, minor physiological fluctuations, or environmental interference such as ambient temperature drift or incidental light exposure. By requiring correlated threshold exceedance across multiple sensor modalities—such as a simultaneous increase in illumination and a loss of contact pressure—the system verifies that an actual environmental or physiological change has occurred. This multi-sensor confirmation logic thereby improves signal integrity, minimizes unnecessary alerts, and ensures that only meaningful, contextually confirmed events trigger data recording or transmission. The approach further enables adaptive confidence scoring, where the magnitude and timing of multiple threshold crossings can be weighted to distinguish true behavioral or environmental events from random sensor noise, thereby improving the accuracy and trustworthiness of recorded data.

### Signal Processing and Event Classification

As used herein: (i) an anomaly means a deviation of a single sensor channel beyond its threshold; (ii) a flagged anomaly means an anomaly recorded without initiating an event cycle; and (iii) a confirmed event means a condition in which data from at least two independent sensor channels exceed their respective thresholds concurrently or within a defined confirmation window, thereby initiating an event cycle.

The microcontroller executes stored firmware instructions that continuously monitor, filter, and interpret sensor signals to detect deviations from expected baseline conditions. During normal operation, baseline reference values are periodically updated to reflect stable environmental and physiological readings while the device is properly worn. The baseline data may include temperature, contact force or capacitance, and ambient light intensity averaged over a defined sampling window (for example, ten to sixty seconds).

Each sensor channel may be sampled at a defined rate, such as 10-100 Hz, and the resulting analog signals may be converted to digital values through the microcontroller's internal analog-to-digital converter. The microcontroller can apply noise-reduction and smoothing algorithms (for example, moving-average or median filtering) to reduce transient interference and isolate meaningful changes.

### Temperature Event Detection

In some embodiments the temperature sensor signal is compared to the rolling baseline. When the sensed temperature changes by 1.8° F. or more within ten seconds or less, the firmware flags a thermal anomaly. The microcontroller then determines whether the change also exceeds a rate-of-change threshold (for example, 0.18° F. per second). If either or both thresholds are met, the firmware can classify the condition as a flagged anomaly and records a timestamped data entry

### Touch and Pressure Event Detection

Signals from the capacitive, resistive, or piezoelectric touch sensors are processed to evaluate both contact state and pressure dynamics. For a capacitive sensor, a change in capacitance of at least 20-30% from baseline indicates a loss or gain of contact. For a resistive sensor, an increase or decrease in resistance corresponding to a pressure change of approximately 0.5 N or greater triggers a contact variation flag. For a piezoelectric element, the microcontroller measures voltage amplitude and rate of change; a voltage transient exceeding approximately 100 m V within 40 milliseconds indicates a mechanical impact or displacement. The system may require that one or more of these conditions persist for a minimum duration (for example, 150-250 milliseconds) before confirmation to avoid false triggers from minor motion or vibration.

### Ambient Light Event Detection

The microcontroller also evaluates illumination data from the ambient light sensor. A deviation—either an increase or a decrease—from the baseline value exceeding a threshold magnitude (for example, ±40% or ±20-50 lux) is classified as an illumination anomaly. When combined with simultaneous contact or temperature changes, the anomalies are verified as a confirmed event or environmental change.

### Event Classification Logic

In some embodiments, all sensor anomalies are evaluated within an event-classification framework executed by the microcontroller. The microcontroller includes a processor, memory, and input/output circuitry coupled to a wireless communication module, such as a Bluetooth Low Energy (BLE) or Wi-Fi transceiver. A flagged anomaly is recorded when a single sensor channel exceeds its predefined threshold. A confirmed event is recorded when two or more independent sensors exceed their respective thresholds concurrently or within a defined temporal window, for example within sixty seconds.

In some embodiments upon detection of a confirmed event, the microcontroller initiates an event cycle in which the processor gathers synchronized sensor readings from all active channels, associates each reading with a corresponding timestamp generated by an internal clock, and assembles a data packet containing the timestamped sensor data and event metadata. The microcontroller then executes a transmission routine stored in memory to either (a) transmit the assembled data packet to an external paired device via the wireless communication module, or (b) store the packet in local nonvolatile memory for later retrieval if communication is unavailable. In some embodiments, the communication module and the microcontroller are integrated within a single system-on-chip (SoC), while in other embodiments, the communication module is a discrete component connected to the microcontroller via a serial peripheral interface (SPI), I²C, or UART communication bus.

In some embodiments to minimize redundant recordings, the firmware employs hysteresis logic. Once an event is confirmed, new events will not be recorded until all sensor values return within a defined tolerance (for example, 70% of the original threshold) for a sustained stabilization period such as 90 seconds. This prevents repeated alerts from small oscillations around the threshold boundary.

### Data Logging and Transmission

In some embodiments during the event cycle, the microcontroller packages the collected sensor data—including event type, timestamp, sensor identifiers, and raw or processed readings—into a structured packet and transmits it to the communication module. If the Bluetooth Low Energy connection is active, the data packet is sent immediately to the paired mobile device. If the connection is unavailable, the communication module stores the packet in local memory with chronological indexing. When communication is re-established, the stored packets are automatically retrieved and transmitted in order, ensuring no loss of continuity in event reporting.

### Wireless Communication and Data Handling

The device includes a communication module configured to transmit data generated by the microcontroller to an external device and, when necessary, to operate autonomously during disconnection periods. In one embodiment, the communication module comprises a wireless transceiver, an onboard controller, and non-volatile memory. The transceiver operates under a Bluetooth Low Energy (BLE) protocol to establish a short-range wireless link with a paired mobile device, such as a smartphone or tablet. Alternative embodiments may employ other low-power communication standards, including Wi-Fi, ZigBee, or proprietary sub-GHz radio. As used herein, an external device includes a paired mobile device such as a smartphone or tablet.

During initialization, the device performs a wireless pairing process with an external mobile device through a secure key exchange routine stored in memory. Once the pairing is successfully established, the microcontroller transfers control of outbound communication to the communication module's internal controller. The communication module includes its own processor, memory, and transceiver circuitry configured to manage packet formatting, connection state, and error handling in accordance with a wireless communication protocol, such as Bluetooth Low Energy (BLE).

The communication module continuously monitors link status and dynamically determines whether to transmit or temporarily store event data. When a confirmed event is generated by the microcontroller, an event packet containing a timestamp, synchronized sensor readings, and classification metadata is transferred to the communication module through an inter-module interface such as SPI, I²C, or UART. If a wireless connection to the paired mobile device is active, the communication module immediately transmits the event packet through a BLE characteristic or data channel. If the link is unavailable or unstable, the packet is temporarily stored in local nonvolatile memory within the device and transmitted automatically once the communication link is reestablished.

In certain embodiments, the communication module periodically transmits a status beacon or acknowledgment signal to confirm successful delivery of each event packet. The microcontroller may update a transmission log in memory based on these acknowledgments to maintain a synchronized record of stored and transmitted events. If the BLE connection is unavailable or interrupted, the communication module automatically writes each generated packet to local non-volatile memory along with an internal sequence number and checksum. This ensures chronological ordering and data integrity during offline operation. The communication module periodically checks for link restoration. Upon re-establishment of the BLE connection, the stored packets are retrieved sequentially, verified for checksum accuracy, and retransmitted in proper order to the paired mobile device. This allows autonomous data retention during extended periods without user intervention or network connectivity.

To preserve data privacy, all stored and transmitted information may be encrypted using symmetric or asymmetric cryptographic algorithms (for example, AES-256 or RSA-2048). The microcontroller or the communication controller may perform on-device encryption prior to transmission. In one embodiment, encryption keys are negotiated during initial pairing with the mobile device and refreshed at predetermined intervals to prevent unauthorized access.

In some embodiments when the paired mobile device receives the transmitted data, a companion mobile application immediately relays the encrypted packet to a secure cloud server over an internet connection without storing the packet in the mobile device's persistent memory. This design prevents retention of sensitive data on local consumer hardware and allows server-side data management under controlled access permissions. In the absence of internet connectivity, the mobile application temporarily buffers packets in memory and transmits them to the cloud upon reconnection.

The communication architecture thus ensures three-tier reliability:
1. On-device storage within the communication module during offline states;
2. Short-range wireless transfer to the mobile device when local connectivity is restored; and
3. Secure cloud synchronization for permanent archival, analytics, or authorized review.

To further maintain chronological integrity, each data packet includes an internal timestamp derived from the microcontroller's real-time clock (RTC) or synchronized mobile device clock. The timestamps enable the cloud platform to reconstruct continuous event timelines, even across temporary disconnections, power cycles, or firmware restarts.

In certain embodiments, prior to activation, the system enforces a digital mutual-consent protocol between two registered users. Each user must provide authenticated consent through a mobile or web interface before the device may operate in data-collection mode. The consent data may be cryptographically signed and stored in association with the device identifier. The microcontroller may verify the presence of valid consent tokens before enabling any sensor-based recording or transmission functions, ensuring that operation occurs exclusively within a consensual, user-configured environment.

In other embodiments, when a user voluntarily removes the device, the mobile application provides an interface for designating the removal as a neutral event. Upon receiving this input, the microcontroller records the removal without generating a flagged or confirmed alert. Similarly, if the device autonomously detects a combination of contact loss, light exposure, or temperature drop consistent with a voluntary removal, such events are classified as neutral entries. The paired mobile device does not store or retain behavioral data beyond the duration of an active session, ensuring compliance with privacy constraints and data-minimization principles.

Together, the microcontroller and communication module function cooperatively to provide self-contained, autonomous operation, reliable offline storage, and secure, consent-based data transmission. This configuration ensures continuous monitoring, privacy preservation, and accurate data synchronization across mobile and cloud environments, even in the absence of constant wireless connectivity.

### Optional Sensor and Module Integration

In certain embodiments, the body-worn device further comprises one or more supplemental sensor modules that expand the range of detectable physiological and environmental parameters while maintaining compact housing dimensions and low-power operation.

### Onboard Camera

In certain embodiments, the housing further includes an onboard camera oriented to capture still images or video sequences under control of the microcontroller. The camera may include a solid-state image sensor such as a CMOS or CCD module with integrated optics and low-light capability. Upon detection of a confirmed event, as defined by concurrent anomalies from at least two sensors, the microcontroller activates the camera to capture one or more frames or a short video sequence. The image data are temporarily stored in encrypted form or non encrypted form within local memory of the communication module and subsequently transmitted through the paired mobile device to a secure cloud server. The camera activation sequence may include safeguards such as requiring a confidence score for the confirmed event above a stored threshold.

The housing may be configured for different anatomical placements. In some embodiments, the housing is designed for insertion within a vaginal canal, while in alternative embodiments, it is configured for mounting on, around, or adjacent to a male genital region, including the penis or scrotum. In each configuration, sensor positioning and housing geometry are optimized to ensure accurate data acquisition, stable contact, and user comfort while maintaining privacy through data encryption and consent-based activation logic.

In certain embodiments, the housing is formed from a biocompatible elastomeric material such as medical-grade silicone, thermoplastic polyurethane (TPU), or thermoplastic elastomer (TPE). The flexible nature of these materials allows localized deformation in response to physiological movement or tissue expansion, improving the accuracy of pressure, strain, and temperature measurements. The elasticity of the housing also enables conformal contact between the embedded sensors and the surrounding tissue, which enhances thermal coupling for temperature sensing and mechanical coupling for pressure detection.

When the housing is inserted within a vaginal canal, the temperature sensor is positioned and thermally coupled to the housing surface to measure core body temperature with improved accuracy relative to peripheral skin temperature measurements. The material's flexibility allows subtle displacement of the embedded pressure sensors to detect changes in internal pressure or muscular contraction without causing discomfort.

In certain embodiments, the housing is configured for external or semi-enclosing placement around the male genital region. For example, the housing may be shaped as a flexible sheath, wrap, or ring configured to conform to the penile shaft or scrotal surface. The housing may be partially or fully circumferential to provide stable contact with the skin while allowing for natural movement and comfort during wear.

In these embodiments, the housing is composed of a biocompatible and elastomeric material such as medical-grade silicone, thermoplastic polyurethane (TPU), or thermoplastic elastomer (TPE). The flexible and stretchable nature of these materials allows the housing to expand and contract with physiological changes such as tissue swelling, vascular engorgement, or muscle contraction. This deformation is detected by embedded pressure or strain sensors, thereby enabling measurement of dynamic physiological parameters such as erectile rigidity, local pulse wave, or muscular tension.

The material's elasticity also enhances thermal coupling between the housing and the skin, allowing temperature sensors embedded near the inner surface to measure local temperature variations correlated with blood flow or thermoregulatory changes. The housing geometry and surface texture may be further optimized to maintain consistent contact with the skin and to minimize motion artifacts.

In some embodiments, the housing includes an internal liner or microtextured surface to promote stable adhesion without excessive compression. The exterior surface may be hydrophobic or coated with a biocompatible lubricant to improve comfort and wearability. Sensor leads or wireless communication components may be embedded within or routed along the housing wall, maintaining signal integrity while preserving flexibility.

### Definition of Continuous Operation

As used herein, the terms “continuous event timelines,” “continuous sampling,” and “continuous monitoring” do not require uninterrupted or constant data collection. Instead, these terms refer to periodic or near-continuous acquisition and processing of sensor data at intervals sufficiently short to preserve meaningful temporal resolution and real-time responsiveness for the intended application. Depending on sensor type, environmental stability, and power-management configuration, sampling intervals may range from approximately 10 milliseconds to several minutes, such as between 0.01 seconds and 300 seconds. Faster sampling rates may be employed for rapidly changing inputs such as touch, pressure, or motion, while slower sampling intervals may be used for parameters such as temperature or ambient light. In certain embodiments, the microcontroller dynamically adjusts sampling frequency based on detected activity, operating conditions, or battery level to balance responsiveness and energy efficiency. Accordingly, the system achieves continuous functional monitoring and maintains a continuous event timeline even when individual sensors are read intermittently or in a duty-cycled pattern.

### Additional Embodiments

### Touch- and Pressure-Sensing Functionality

In some embodiments, the touch sensor is configured to perform both contact detection and pressure measurement without the need for a separate pressure sensor. When implemented as a resistive touch sensor, variations in applied force cause a measurable change in electrical resistance between conductive layers, allowing the microcontroller to determine both the presence and magnitude of contact pressure. Similarly, when implemented as a piezoelectric force sensor, mechanical deformation of the sensing element generates a voltage proportional to the applied pressure, enabling detection of dynamic or transient force changes. The sensor provides this output data to the microcontroller, which compares the readings to stored baseline values to identify contact loss, pressure increase, or pressure decrease. Deviations exceeding predefined thresholds may be classified as pressure anomalies or events, particularly when correlated with concurrent changes in temperature or ambient light. In alternative embodiments, a dedicated pressure sensor may be incorporated to supplement these measurements, allowing finer resolution or redundancy in force detection.

### Temperature Sensor

For embodiments in which the housing is insertable within a body cavity, the temperature sensor is positioned to detect internal body temperature with improved accuracy relative to peripheral surface measurements. The temperature sensor generates an output signal representative of the measured temperature and provides this signal to the microcontroller for processing. The microcontroller receives and records the temperature data, compares it to stored baseline values, and identifies changes exceeding predefined thresholds. The system may optionally average or calibrate readings to maintain consistent measurement accuracy during extended wear.

### Inertial Measurement Unit (IMU)

In certain embodiments, the device further includes an inertial measurement unit (IMU) integrated within the housing. The IMU may include one or more motion sensors, such as an accelerometer, gyroscope, or magnetometer, configured to detect movement, orientation, or vibration of the housing. The IMU provides motion-related data to the microcontroller, which processes this information to determine whether the device has been moved, repositioned, or disturbed. The microcontroller may compare IMU data with concurrent readings from other sensors to confirm proper placement, identify removal or tampering, or verify the occurrence of an environmental or physiological event.

### Geolocation Module

In some embodiments, the device includes a geolocation module configured to determine the geographic location of the housing. The module receives signals from one or more satellite-based navigation systems and provides corresponding location data to the microcontroller. When activated—such as during or following detection of a confirmed event—the microcontroller may record the geolocation data together with the sensor readings to establish contextual information about the event. The system may further conserve power by enabling the geolocation function only when necessary for data association or event confirmation.

### Physiological Data Generation

In certain embodiments, the microcontroller processes data collected from the temperature, contact, and motion sensors to generate physiological information related to user activity or health. These data may include temperature trends, contact duration, movement patterns, or other derived parameters associated with general physiological states or activity cycles. The processed information is stored in memory or transmitted through the communication module as part of an event record. This arrangement allows the system to monitor and record physiological changes over time while maintaining secure and controlled data handling.

As used herein, the term “external device” includes a paired mobile device and any networked endpoint accessible through the mobile device, such as a cloud server, remote database, or authorized web interface, to which event data are transmitted directly or indirectly via the paired mobile device.

### Baseline Initialization and Averaging Routine

In some embodiments, the device includes a baseline initialization sequence that establishes reference values for each active sensor channel prior to continuous monitoring. Upon user activation, such as by pressing an on-housing control button, the device powers on and begins an environmental stabilization period lasting approximately sixty (60) seconds. This delay allows the housing, sensors, and surrounding environment to reach thermal and mechanical equilibrium before baseline acquisition begins. During this period, no data are stored or transmitted.

After the stabilization delay, the microcontroller automatically initiates a baseline configuration routine. Each active sensor channel, including temperature, contact or pressure, and ambient-light sensors, is sampled at a defined rate corresponding to approximately five (5) to one hundred (100) readings per second (5-100 Hz), which is equivalent to one reading every 0.01 to 0.2 seconds depending on the sensor type and temporal resolution required. Temperature sensors may be sampled at lower rates, for example one (1) to five (5) Hz (one reading every 0.2-1 second), whereas pressure or touch sensors may be sampled at higher rates such as fifty (50) to one hundred (100) Hz (one reading every 0.01-0.02 seconds) to capture rapid changes in mechanical contact.

The microcontroller collects these samples for an averaging interval of approximately thirty (30) to one hundred twenty (120) seconds following the stabilization delay. During this interval, the firmware computes a baseline reference value for each channel by averaging all valid samples within the window. Outlier rejection may be applied by discarding samples deviating more than a defined percentage, for example ±15 percent, from the median. In alternative embodiments, the firmware applies a rolling-median or exponentially weighted moving-average filter to provide temporal smoothing and to suppress transient fluctuations caused by motion, minor repositioning, or local temperature drift.

The resulting baseline set defines a temperature baseline representing the steady thermal state of the housing in contact with adjacent tissue, a pressure or contact baseline representing the normal mechanical coupling between the housing and surrounding anatomy, and a light-intensity baseline representing the nominal illumination level when the housing is properly positioned and shielded from external light.

Each baseline value is stored in non-volatile memory along with a timestamp and session identifier. Subsequent sensor readings are continuously compared to these stored baseline references to determine absolute or percentage deviations. Deviations exceeding one or more predefined thresholds, for example ±1.8° F. for temperature, ±25 percent for contact, or ±40 percent for illumination, are classified as anomalies or potential events according to the event-classification framework.

In some embodiments, the device automatically re-establishes baseline references after prolonged operation or may allow the user to initiate recalibration through the paired mobile device. Each recalibration sequence follows the same stabilization and averaging procedure described above, thereby ensuring consistent environmental normalization and reliable detection performance throughout the device's operational life.

### Unique User Profile

In certain embodiments, the housing further includes a plurality of pressure sensors disposed on or embedded within an interior or exterior portion of the housing and equally spaced about an axis. The arrangement provides uniform radial distribution of pressure measurement points regardless of whether the housing is cylindrical, ring-shaped, or otherwise rotationally symmetric. Each sensor may comprise a piezoresistive, capacitive, or piezoelectric element in electrical communication with analog input channels of the microcontroller.

During initializing baseline values, the microcontroller samples each pressure sensor at approximately 50-100 Hz for a baseline interval of 30-120 seconds, calculates an average baseline pressure value per sensor, and stores the resulting set of baseline values as a user-specific pressure profile in non-volatile memory. The firmware then causes the communication module to transmit the stored pressure profile to a secure cloud server via a paired mobile device over a Bluetooth Low Energy (BLE) link, where the data are archived for subsequent baseline verification, performance validation, or adaptive threshold adjustment.

In some embodiments, the paired mobile device may utilize transient memory, such as volatile random-access memory (RAM), to temporarily store data received from the body-worn device's microcontroller or communication module prior to forwarding the data to a remote server or cloud platform. This configuration ensures that no persistent local copy of sensitive data remains on the mobile device after transmission. In alternative embodiments, the mobile device may employ non-transient memory, such as flash storage or other forms of non-volatile memory, to locally store data received from the body-worn device's microcontroller or communication module. Such non-transient storage may be used for redundancy, offline archival, or delayed synchronization purposes, maintaining chronological integrity of the data during periods without network connectivity.

In some embodiments, data generated by the body-worn device is encrypted in transit and/or at rest using industry-standard cryptography. For symmetric encryption, the system may employ AES-128/192/256 in an authenticated mode (e.g., AES-GCM or AES-CCM) or, in other embodiments, ChaCha20-Polyl305. For asymmetric cryptography, the system may utilize RSA-2048/3072/4096 (e.g., RSA-OAEP for encryption, RSASSA-PSS for signatures) and/or elliptic-curve schemes such as ECDH/ECDSA on NIST P-256/P-384, X25519 (key agreement), and Ed25519 (signatures). Session keys may be negotiated via TLS 1.3 or DTLS 1.3, or via Bluetooth LE Secure Connections using ECDH; keys may be derived using HKDF and authenticated with HMAC-SHA-256/384/512. For data at rest on the device, mobile handset, or cloud storage, encryption may use AES-XTS, AES-GCM, or platform file-based encryption with hardware acceleration where available. Digital integrity and origin authentication may be provided by HMAC or digital signatures (ECDSA/Ed25519/RSASSA-PSS). In some embodiments, hardware-backed key storage (e.g., Secure Element, Trusted Execution Environment, or platform keystore) is used to protect long-term keys and perform crypto operations; keys may be rotated on a scheduled basis and ephemeral session keys used for payload encryption. The system may optionally enforce end-to-end encryption between the body-worn device and a cloud endpoint, with the paired mobile device acting solely as a pass-through transport. Implementations may target relevant compliance baselines (e.g., FIPS 140-3-validated modules) without limiting the scope of the invention.

What is claimed is:

1. A body-worn device comprising:
(a) a housing adapted to be mounted within or adjacent to a genital region of a user;
(b) at least one touch sensor selected from the group consisting of a capacitive touch sensor, resistive touch sensor, or piezoelectric force sensor, the at least one touch sensor being capable of detecting physical contact;
(c) a temperature sensor;
(d) an ambient-light sensor operable to measure illumination levels surrounding the housing;
(e) a microcontroller comprising a processor, a memory, and input/output circuitry, the microcontroller further programmed to:
(i) upon user activation, delay data collection for a stabilization period of about 60 seconds;
(ii) during a baseline interval of about 30 to 120 seconds following the stabilization period, sample the temperature sensor at 1-5 Hz, the touch or pressure sensor at 50-100 Hz, and the ambient-light sensor at 5-50 Hz; compute per-channel baselines using averaging with outlier rejection at ±15 percent; and store the baselines in non-volatile memory;
(iii) classify a confirmed event only when data from at least two sensors concurrently or sequentially exceed their respective thresholds, the thresholds comprising at least one of: a temperature change of ≥1.8° F. within 10 seconds, an ambient-light deviation of ≥40 percent from baseline within 2 seconds, or a touch or pressure deviation of ≥25 percent from baseline, wherein such exceedances occur within a confirmation window of 60 seconds; and
(iv) inhibit subsequent confirmed events until all sensor readings return within 70 percent of their thresholds for at least 90 seconds;
(v) during a confirmed event, execute instructions for data collection, apply internal timestamps, and generate an outbound transmission packet;
(f) a communication module comprising a wireless transceiver, a memory, and a controller, the communication module programmed to:
(i) establish a short-range wireless connection with a paired mobile device through the wireless transceiver;
(ii) transmit data generated by the microcontroller to the paired mobile device over the wireless connection;
(iii) store the data in the memory when the wireless connection is unavailable at the time of transmission; and
(iv) upon restoration of the wireless connection, retrieve the stored data from the memory and transmit the data to the paired mobile device; and
(g) a power supply integrated within the housing and providing electrical energy to the microcontroller and the communication module;
wherein the microcontroller and the communication module cooperate to enable autonomous operation of the device by storing event data in the memory when the communication module is disconnected from the paired mobile device and transmitting the stored event data to the paired mobile device upon re-establishment of the connection.

2. The body worn device of claim 1, wherein the device further comprises an inertial measurement unit (IMU) including at least one of an accelerometer, a gyroscope, or a magnetometer.

3. The body worn device of claim 2, further comprising a geolocation module including a receiver for signals from a satellite-based navigation system.

4. The body-worn device of claim 1, wherein the housing is insertable within a vaginal canal of the user, the temperature sensor positioned to detect core body temperature with improved accuracy relative to peripheral skin temperature measurements.

5. The body-worn device of claim 4, wherein the housing is positioned within a vaginal canal of the user, the temperature sensor and microcontroller cooperating to generate physiological data associated with sexual activity, fertility cycles, or urogenital health.

6. The body-worn device of claim 1, further comprising an onboard camera disposed within the housing, the camera operable to capture still images or video sequences upon activation by the microcontroller in response to detection of a confirmed event.

7. The body-worn device of claim 6, wherein the housing is adapted to be mounted on, around, or adjacent to a male genital region, including at least one of the penis or the scrotum.

8. The body-worn device of claim 4, further comprising an onboard camera disposed within the housing, the camera operable to capture still images or video sequences upon activation by the microcontroller in response to detection of a confirmed event.

9. The body-worn device of claim 1, wherein the touch sensor is further configured to operate as a pressure sensor, the touch sensor being operable to detect pressure exerted against the housing at its mounted location and to transmit corresponding pressure data to the microcontroller for processing.

10. The body-worn device of claim 9, wherein the housing further comprises at least four pressure sensors disposed at substantially quadrantal positions about an exterior surface of the housing, each pressure sensor configured to obtain a baseline pressure value during a baseline interval, and wherein the microcontroller is further programmed to generate a unique user-specific pressure profile based on the set of baseline pressure values corresponding to the four sensors and to cause the communication module to transmit the user-specific pressure profile to a secure cloud server via the paired mobile device.

11. A method of operating a body-worn device having a housing adapted for insertion within a vaginal cavity of a user, the method comprising:
(a) establishing wireless pairing of the device with a mobile device over a Bluetooth Low Energy (BLE) connection;
(b) initializing baseline values for:
(i) at least one touch sensor selected from the group consisting of a capacitive touch sensor, a resistive touch sensor, and a piezoelectric force sensor;
(ii) a temperature sensor; and
(iii) an ambient light sensor operable to measure illumination levels surrounding the housing;
(c) continuously sampling signals from the touch sensor, the temperature sensor, and the ambient light sensor with a microcontroller, and comparing the sampled signals to the baseline values and predefined thresholds stored in memory;
(d) detecting one or more anomalies comprising at least one of:
(i) a temperature change relative to the baseline,
(ii) loss of contact at the touch sensor, or
(iii) exposure to ambient light;
(e) classifying the anomalies, wherein a single anomaly is flagged without confirmation, and wherein concurrent or temporally proximate anomalies comprising at least two of the anomalies are verified as a confirmed event;
(f) upon classification of a confirmed event, activating an onboard camera to capture visual documentation comprising still images or a video sequence;
(g) transmitting the data via the BLE connection to the paired mobile device;
(h) relaying the data from the mobile device to a cloud server without storing the data locally on the mobile device; and
(i) when internet connectivity is unavailable, storing the data in memory of the body-worn device and automatically transmitting the stored data to the cloud server upon restoration of connectivity.

12. The method of claim 11, further comprising encrypting the data and storing it in memory of the communication module when no wireless connection is available, maintaining chronological data integrity across offline periods, reboots, or delayed synchronizations, and transmitting the stored data upon re-establishment of the wireless connection.

13. The method of claim 11, further comprising enabling the user to designate a voluntary removal event through an input on the paired mobile device or another external interface prior to removal of the body-worn device, wherein the microcontroller records the designated removal as a neutral, non-alerting entry.

14. The method of claim 11, further comprising logging voluntary device removal events as neutral entries based on detection of at least one of contact loss, light exposure, or temperature change by the sensors, wherein such removal events are not classified as flagged or confirmed anomalies, and wherein the paired mobile device does not persistently store behavioral data beyond the duration of an active session.

15. The method of claim 11, further comprising initiating capture of visual documentation if a behavioral event is classified as confirmed and satisfies a stored confidence threshold.

16. The method of claim 15, wherein the visual documentation is encrypted, stored locally in device memory, and transmitted through a cloud platform or mobile application.

17. The method of claim 11, wherein the body-worn device is permitted only after at least two users have digitally provided mutual consent through a system interface, such that operation occurs exclusively in a consensual, user-configured environment.

18. A method of operating a body-worn device adapted to be mounted on, around, or adjacent to a male genital region, including at least one of the penis or the scrotum, the method comprising:
(a) establishing wireless pairing of the device with a mobile device over a Bluetooth Low Energy (BLE) connection;
(b) initializing baseline values for:
(i) at least one touch sensor selected from the group consisting of a capacitive touch sensor, a resistive touch sensor, and a piezoelectric force sensor;
(ii) a temperature sensor; and
(iii) an ambient light sensor operable to measure illumination levels surrounding the housing;
(c) continuously sampling signals from the touch sensor, the temperature sensor, and the ambient light sensor with a microcontroller, and comparing the sampled signals to the baseline values and predefined thresholds stored in memory;
(d) detecting one or more anomalies comprising at least one of:
(i) a temperature change relative to the baseline,
(ii) loss of contact at the touch sensor, or
(iii) exposure to ambient light;
(e) classifying the anomalies, wherein a single anomaly is flagged without confirmation, and wherein concurrent or temporally proximate anomalies comprising at least two of the anomalies are verified as a confirmed event;
(f) upon classification of a confirmed event, activating an onboard camera to capture visual documentation comprising still images or a video sequence;
(g) transmitting the data via the BLE connection to the paired mobile device;
(h) relaying the data from the mobile device to a cloud server without storing the data locally on the mobile device; and
(i) when internet connectivity is unavailable, storing the data in memory of the body-worn device and automatically transmitting the stored data to the cloud server upon restoration of connectivity.

19. The method of claim 18, further comprising encrypting the data and storing it in memory of the communication module when no wireless connection is available, maintaining chronological data integrity across offline periods, reboots, or delayed synchronizations, and transmitting the stored data upon re-establishment of the wireless connection.

20. The method of claim 18, wherein the body-worn device is permitted only after at least two users have digitally provided mutual consent through a system interface, such that operation occurs exclusively in a consensual, user-configured environment.
